(19) United States
(12) Patent Application Publication
(10) Pub. No.: US 2023/0366895 A1
(43) Pub. Date: Nov. 16, 2023

(54) REAGENT FOR DETECTING LIPOPROTEIN PARTICLE CONCENTRATION AND USING METHOD THEREOF

(71) Applicant: MEDICAL SYSTEM BIOTECHNOLOGY CO., LTD., Ningbo City, Zhejiang Province (CN)

(72) Inventors: Bingde ZOU, Ningbo City, Zhejiang Province (CN); Yi WANG, Ningbo City, Zhejiang Province (CN); Jianghua JIA, Ningbo City, Zhejiang Province (CN)

(21) Appl. No.: 18/029,110

(22) PCT Filed: Feb. 4, 2021
(86) PCT No.: PCT/CN2021/075263

§ 371 Date: Mar. 29, 2023

(30) Foreign Application Priority Data
Oct. 27, 2020 (CN) 202011160074.3

(51) Int. Cl. G01N 33/92; G01N 15/06

(52) U.S. Cl. CPC G01N 33/92; G01N 15/06; G01N 2015/0693

(57) ABSTRACT

The present invention provides a reagent for detecting a lipoprotein particle concentration, comprising the following components: a diluent, a density liquid and a buffer solution, where the density liquid contains an alcohol substance with a concentration of 0.1% to 50%. The present invention relates to the field of in-vitro diagnoses. The present invention provides a reagent for detecting a lipoprotein particle concentration, which can not only detect the lipoprotein particle concentration separately but also detect a cholesterol sub-component of the lipoproteins at the same time in cooperation with other instruments and reagents, thereby combining two processes into one process and saving time and costs. Therefore, the present invention has high commercial value and can be widely applied to clinical detections and scientific researches.

### TECHNICAL FIELD

The present invention relates to the field of in vitro diagnoses and in particular to a reagent for detecting a particle concentration of a lipoprotein component in a serum sample.

### BACKGROUND

It is well known that, based on different lipoprotein densities, the lipoproteins can be divided into a chylomicron (CM), a very low-density lipoprotein (VLDL), an intermediate density lipoprotein (IDL), a low-density lipoprotein (LDL), and a high-density lipoprotein (HDL). Clinically, cholesterol contents of various lipoproteins are generally measured to guide diagnosis. At present, in addition to total cholesterol (T-CH), LDL-C and HDL-C are also generally determined clinically, where excessively high LDL-C content may easily result in atherosclerosis which is called bad cholesterol, whereas HDL-C has protection for blood vessels and is called good cholesterol. However, a recent research shows that both LDL-C and HDL-C have heterogeneity and their clinical significances are also not completely same. For example, LDL-C can be divided into many subcomponents: small and dense low density lipoprotein cholesterol (sdLDL-C), which easily results in atherosclerosis due to its compact structure, called B type; and large and light low density lipoprotein cholesterol (LLDL-C) which is less able to result in atherosclerosis, called A type. The HDL-C can also be divided into at least two main subcomponents, for example, large and light HDL2 particles and small and dense HDL3 particles. Some research evidences show that compared with measuring only HDL, measuring HDL subcomponents can better reflect the risk of cardiovascular diseases.

Traditionally, the detection of lipoproteins is performed by detecting a content of cholesterol in lipoprotein particles. Due to technical reasons, the lipoprotein particle concentration, such as VLDL particle concentration, LDL particle concentration and HDL particle concentration is seldom detected directly. In fact, these detections of the lipoprotein particle concentration reflect a level of in-vivo lipoprotein from another angle, and have the effect of evaluating the risk of the cardiovascular diseases.

One method is a nuclear magnetic resonance method. In this method, by using a type of hydrogen on the methyl of lipoproteins, through complex algorithm, the particle concentration of the lipoproteins and its subcomponents can be obtained. This method has high technical requirements with expensive instruments involved, and high requirements are also proposed for the operators. Therefore, the method is more suitable for scientific researches. Hence, it is difficult to promote it in the clinical tests.

United States patents with the publication numbers U.S. Pat. Nos. 5,284,773A and 5,633,168A disclose a technology relating to vertical auto profile (VAP), where ultra-speed centrifugation is performed for lipoproteins to detect the cholesterol contents of different lipoproteins. The invention patent with the US publication number U.S. Pat. No. 9,239,280B2 discloses a technology relating to vertical lipoprotein profile, where ultra-speed centrifugation is performed for lipoproteins to detect the particle concentrations of different lipoproteins, which is similar to the nuclear magnetic resonance method. Chinese patent with the publication number CN110108673A combines above several patents to disclose a method of detecting a cholesterol content of different lipoproteins and a particle concentration of the lipoproteins at the same time.

Due to limitation of the ultracentrifugation system in the above patents, various lipoproteins cannot be fully separated and thus interference may occur between adjacent lipoproteins. When the detection system is a VLP detection system (the instrument used is called a blood lipid particle tester), the detection of scattered light signals is related to the size of the lipoprotein particles, and when the large and small particles are not fully separated, the influence of the large particles on the small particles will be more obvious. Especially, LDL-P (relatively small) is extremely susceptible to interference of IDL-P (relatively large) and VLDL-P (large particles), and such interference is more obvious when the concentration of triglyceride in the sample is high. However, these items will be significantly subjected to positive interference for there are some chylomicron particles in the high-triglyceride sample. Its interference effects on other lipoproteins are, in an order of effect size, VLDL-P, IDL-P, LDL-P. In severe case, the precisions of the items LDL-P, IDL-P, VLDL-P and the like are affected. Hence, in the patent U.S. Pat. No. 9,239,280B2 which discloses separately detecting the lipoprotein particle concentration or in the patent CN110108673A which discloses detecting the cholesterol content of the lipoproteins and the particle concentration of the lipoproteins at the same time, poor precision and high susceptibility to high triglyceride sample are exhibited.

### SUMMARY

The technical problem to be solved by the present invention is that: a density gradient separation reagent is provided, where performing density gradient ultracentrifugation by using the reagent can effectively improve a precision of a lipoprotein particle concentration detection system, while reducing an interference of a high triglyceride sample. The present invention can be combined with other patents (e.g. background technologies) such that a lipoprotein particle concentration can be detected separately and a cholesterol subcomponent of lipoproteins can be detected at the same time, thus combining two detections into one detection and saving time and costs.

The technical solution used by the present invention is to provide a reagent for detecting a lipoprotein particle concentration, where the reagent comprises the following components:

a diluent, a density liquid and a buffer solution, where the density liquid contains an alcohol substance with a concentration of 0.1% to 50%, and a volumetric ratio of the diluent to the density liquid to the buffer solution is 1: (35-45): (110-130).

As a preferred scheme, the number of the carbon atoms contained in the alcohol substance is 1, 2, 3 or 4.

As a preferred scheme, the alcohol substance is one or more of methanol, ethanol, ethylene glycol, n-propanol, isopropanol, propylene glycol, n-butanol, isobutanol and tertiary butanol.

As a preferred scheme, the concentration of the alcohol substance is 1% to 5%.

As a preferred scheme, the density liquid further comprises NaCl and EDTA.

As a preferred scheme, the typical formulas (adjust to a designated density by using NaCl) of the density liquid comprise but not limited to:
formula A: NaCl, 0.1 mM EDTA, 0.1% ethanol, density 1.05 g/cm³;
formula B: NaCl, 0.1 mM EDTA, 25% ethanol, density 0.98 g/cm³;
formula C: NaCl, 0.1 mM EDTA, 5% ethanol, density 1.05 g/cm³;
formula D: NaCl, 0.1 mM EDTA, 1% isopropanol, density 1.05 g/cm³;
formula E: NaCl, 0.1 mM EDTA, 5% isopropanol, density 1.03 g/cm³;
formula F : NaCl, 0.1 mM EDTA, 0.5% n-propanol, density 1.05 g/cm³;
formula G: NaCl, 0.1 mM EDTA, 0.5% n-butanol, density 1.05 g/cm³;
formula H: NaCl, 0.1 mM EDTA, 1% isobutanol, density 1.05 g/cm³;
formula L NaCl, 0.1 mM EDTA, 0.5% tertiary butanol, density 1.05 g/cm³;
formula J: NaCl, 0.1 mM EDTA, 0.1% ethylene glycol, density 1.05 g/cm³;
formula K:NaCl, 0.1 mM EDTA, 0.5% propylene glycol, density 1.05 g/cm³.

As a preferred scheme, the diluent is one of a KBr solution, a NaBr solution and a sucrose solution, and a concentration of the diluent is 1.21 g/cm³.

As a preferred scheme, the buffer solution may be one of a tris (hydroxymethyl) aminomethane hydrochloride (Tris-HCl) buffer solution, a phosphate buffer solution, a 4-(2-hydroxyethyl)piperazine-1-ethanesulfonic acid hemisodium salt (HEPES) buffer solution, a 3-(N-morpholino) ethanesulfonic acid (MOPS) buffer solution, and an N-tris(hydroxymethyl)methyl-2-aminoethanesulfonic acid (TES) buffer solution.

As a preferred scheme, provided is a using method of the reagent which mainly comprises the following steps:

I. diluting a serum sample by using the diluent and fully mixing the serum sample to uniformity to obtain a diluted sample, and adding the density liquid to a centrifugal tube; taking a part of the diluted sample and slowly adding the diluted sample to the centrifugal tube through the bottom of the centrifugal tube to obtain a mixed solution;

II. placing the treated mixed solution centrifugal tube into an ultracentrifuge for centrifugation and obtaining a stratification reagent after the centrifugation, wherein a rotor used in the ultracentrifuge is a vertical rotor;

III. taking out the above stratification reagent and placing the reagent into a blood lipid particle tester for detection, and adding the buffer solution to the blood lipid particle tester; during detection, performing operation as instructed in the use manual of the tester; ranking the centrifuged lipoproteins based on density and enabling the centrifuged lipoproteins to sequentially go through a light scattering detector which sequentially records received signals, and finally by using the tester, calculating the particle concentration of the lipoproteins in the sample and thus completing test.

As a preferred scheme, in step II, the specific process of the centrifugation is rotation speed 60000 to 70000 rpm, temperature 20 to 25° C., acceleration=6, deceleration=6, and centrifugation time 20 to 30 minutes.

The object of the present invention is to reduce the interference of high triglyceride in sample on detection. Through repeated formula adjustments, good effects are not obtained at the early stage. After an alcohol substance is added to the density liquid by the inventor, it is unexpectedly found that the addition of the alcohol substance can greatly improve the precision of detection of the high triglyceride sample and have obvious effect in reduction of interference caused by triglyceride. There is no special requirement for the alcohol substance added to the density liquid in the present invention, as long as it is an alcohol substance. The inventor tried a large number of alcohol substances with different concentrations. Generally, when the number of carbon atoms contained in alcohol is 1 to 4, good effect can be achieved. These alcohol substances comprise but not limited to: methanol, ethanol, ethylene glycol, n-propanol, isopropanol, propylene glycol, n-butanol, isobutanol and tertiary butanol and the like. When the concentration is between 0.1% and 50%, good effect can be achieved. When the concentration of the alcohol is excessively high, it is difficult to adjust density. Thus, the more commonly concentration is 0.5% to 25%, most commonly, 1% to 5%. The density liquid is used to adjust density during a detection process, that is, used in cooperation with the diluent, such that the sample before centrifugation is formed into upper and lower layers with different densities. In this way, continuous density gradients can be formed after centrifugation to facilitate separation of lipoproteins based on density gradients.

According to detection operation steps, the sample is to be firstly diluted, that is, the serum sample is diluted 40 times or another number of times by using the diluent, which is related to detection sensitivity. If the detection sensitivity is high, the sample can be diluted more times. A typical dilution method is to add 1950 ul of diluent into 50 ul of serum and stir them to uniformity. Further, one 5.0 ml Polyallomer quick-seal tube (a centrifugal tube, or a centrifugal tube with similar function) is taken, and then 3800 ul of density liquid is added, where the density liquid may be any formula reagent mentioned above. Next, 1200 ul of diluted sample is taken and then slowly added through the bottom of the centrifugal tube. The treated sample is placed into an ultracentrifuge for centrifugation. Vertical rotor, for example, VTi-65.2 rotor, must be used to centrifuge 16 samples at one time. Of course, another type of vertical rotors may also be used. Based on different density liquids, different centrifugation procedures are prepared. For example, if the density liquid has a smaller density, the centrifugation time will be shortened. The most common centrifugation time is 30 minutes, and another time such as 28 minutes or 25 minutes or the like may also be used. The rotation speed of the centrifugation is usually set to 65000 rpm, the centrifugation temperature is controlled to about 23° C., acceleration=6 and deceleration=6. The centrifuged sample must be taken out of the centrifuge with great care to prevent violent vibration affecting the centrifuged sample. Then, the sample is tested by using a blood lipid particle tester. During test, the tester needs a corresponding buffer solution so as to enable the entire test process to have a proper buffer environment, because excess acidity or basicity may affect the test result. Usually, there is no special requirement for the buffer solution as long as it can provide a relatively neutral buffer environment. The following buffer solutions may be selected: a tris (hydroxymethyl) amino methane hydrochloride (Tris-HCl) buffer solution, a phosphate buffer solution, a 4-(2-hydroxyethyl)piperazine-1-ethanesulfonic acid hemisodium salt (HEPES) buffer solution, a 3-(N-morpholino) ethanesulfonic acid (MOPS) buffer solution, and an N-tris(hydroxymethyl)methyl-2-aminoethanesulfonic acid (TES) buffer solution. During detection, operations are performed by referring to the use manual of the instrument. The main principle is as follows: in an appropriate buffer environment, the centrifuged lipoproteins are ranked based on density and sequentially subjected to the light scattering detector which records the received signals in sequence. Since the signal intensity is related to the particle concentration of the lipoproteins running through the light scattering detector at a time point, the particle concentration of the lipoproteins in the sample can be reflected. Through a dedicated software and proper calibration, the detail information of the particle concentration of each lipoprotein can be obtained finally. The test items comprise: HDL particle concentration (HDL-P), LDL particle concentration (LDL-P), IDL particle concentration (IDL-P), Lpa particle concentration (Lpa-P), VLDL particle concentration (VLDL-P). The detailed principle may be referred to U.S. Pat. No. 9,239,280. The reagent of the present invention can be used on the above blood lipid particle tester, or in a detection system described in CN110108673A, where the detection system can detect a cholesterol content of the lipoproteins and the particle concentration of the lipoproteins at the same time.

### EMBODIMENTS

The technical scheme of the present invention will be fully and clearly described below. Apparently, the embodiments described herein are only some embodiments of the present invention rather than all embodiments. All other embodiments obtained by those of ordinary skill in the art based on the embodiments of the present invention without making creative work shall fall within the scope of protection of the present invention.

### Embodiment 1

Reagent preparation and detection steps

50 ul of serum was taken and 1950 ul of diluent was added (see Table 1) and then stirred to uniformity. One 5.0 ml Polyallomer quick-seal tube was taken and 3800 ul of density liquid was added (see Table 1) and then 1200 ul of diluted serum sample was slowly added through the bottom. The centrifugal tube was placed into the rotor VTi-65.2 and centrifuged using a Beckman ultracentrifuge. Parameters: rotation speed=65000rpm, time (see Table 1), temperature=23° C., acceleration=6 and deceleration=6.

The centrifuged sample was carefully transferred to the lipoprotein particle concentration tester for detection. During the detection, the buffer solution used is a 20 mM phosphate buffer solution with a pH 7.0.

TABLE 1
Formulas of different combinations

| Serial | Diluent | | Density liquid | | Centrifugation |
|---|---|---|---|---|---|
| number | Component | Density(g/ml) | Component | Density(g/ml) | time |
| Control | KBr | 1.21 | NaCl, 0.1 mM EDTA | 1.05 | 30 min |
| formula | | | | | |
| Formula 1 | KBr | 1.21 | NaCl, 0.1 mM EDTA, | 1.05 | 30 min |
| | | | 0.1% ethanol | | |
| Formula 2 | KBr | 1.21 | NaCl, 0.1 mM EDTA, 0.5% | 1.05 | 30 min |
| | | | ethanol | | |
| Formula 3 | KBr | 1.21 | NaCl, 0.1 mM EDTA, 1% | 1.05 | 30 min |
| | | | ethanol | | |
| Formula 4 | KBr | 1.21 | NaCl, 0.1 mM EDTA, 5% | 1.03 | 28 min |
| | | | ethanol | | |
| Formula 5 | KBr | 1.21 | NaCl, 0.1 mM EDTA, 25% | 0.98 | 25 min |
| | | | ethanol | | |
| Formula 6 | KBr | 1.21 | NaCl, 0.1 mM EDTA, 50% | 0.95 | 22 min |
| | | | ethanol | | |
| Formula 7 | KBr | 1.21 | NaCl, 0.1 mM EDTA, 0.1% | 1.05 | 30 min |
| | | | isopropanol | | |
| Formula 8 | KBr | 1.21 | NaCl, 0.1 mM EDTA, 0.5% | 1.05 | 30 min |
| | | | isopropanol | | |
| Formula 9 | KBr | 1.21 | NaCl, 0.1 mM EDTA, 1% | 1.05 | 30 min |
| | | | isopropanol | | |
| Formula | KBr | 1.21 | NaCl, 0.1 mM EDTA, 5% | 1.03 | 28 min |
| 10 | | | isopropanol | | |
| Formula | KBr | 1.21 | NaCl, 0.1 mM EDTA, 25% | 0.98 | 25 min |
| 11 | | | isopropanol | | |
| Formula | KBr | 1.21 | NaCl, 0.1 mM EDTA, 50% | 0.95 | 22 min |
| 12 | | | isopropanol | | |
| Formula | KBr | 1.21 | NaCl, 0.1 mM EDTA, 0.1% n- | 1.05 | 30 min |
| 13 | | | propanol | | |
| Formula | KBr | 1.21 | NaCl, 0.1 mM EDTA, 0.5% n- | 1.05 | 30 min |
| 14 | | | propanol | | |
| Formula | KBr | 1.21 | NaCl, 0.1 mM EDTA, 1% n- | 1.05 | 30 min |
| 15 | | | propanol | | |
| Formula | KBr | 1.21 | NaCl, 0.1 mM EDTA, 5% n- | 1.03 | 28 min |
| 16 | | | propanol | | |
| Formula | KBr | 1.21 | NaCl, 0.1 mM EDTA, 25% n- | 0.98 | 25 min |
| 17 | | | propanol | | |
| Formula | KBr | 1.21 | NaCl, 0.1 mM EDTA, 50% n- | 0.95 | 22 min |
| 18 | | | propanol | | |
| Formula | KBr | 1.21 | NaCl, 0.1 mM EDTA, 0.1% n- | 1.05 | 30 min |
| 19 | | | butanol | | |
| Formula | KBr | 1.21 | NaCl, 0.1 mM EDTA, 0.5% n- | 1.05 | 30 min |
| 20 | | | butanol | | |
| Formula | KBr | 1.21 | NaCl, 0.1 mM EDTA, 1% n- | 1.05 | 30 min |
| 21 | | | butanol | | |
| Formula | KBr | 1.21 | NaCl, 0.1 mM EDTA, 5% n- | 1.03 | 28 min |
| 22 | | | butanol | | |
| Formula | KBr | 1.21 | NaCl, 0.1 mM EDTA, 25% n- | 0.98 | 25 min |
| 23 | | | butanol | | |
| Formula | KBr | 1.21 | NaCl, 0.1 mM EDTA, 50% n- | 0.95 | 22 min |
| 24 | | | butanol | | |
| Formula | KBr | 1.21 | NaCl, 0.1 mM EDTA, 0.1% | 1.05 | 30 min |
| 25 | | | isobutanol | | |
| Formula | KBr | 1.21 | NaCl, 0.1 mM EDTA, 0.5% | 1.05 | 30 min |
| 26 | | | isobutano1 | | |
| Formula | KBr | 1.21 | NaCl, 0.1 mM EDTA, 1% | 1.05 | 30 min |
| 27 | | | isobutanol | | |
| Formula | KBr | 1.21 | NaCl, 0.1 mM EDTA, 5% | 1.03 | 28 min |
| 28 | | | isob uta nol | | |
| Formula | KBr | 1.21 | NaCl, 0.1 mM EDTA, 25% | 0.98 | 25 min |
| 29 | | | isobutanol | | |
| Formula | KBr | 1.21 | NaCl, 0.1 mM EDTA, 50% | 0.95 | 22 min |
| 30 | | | isobutanol | | |
| Formula | KBr | 1.21 | NaCl, 0.1 mM EDTA, 0.1% | 1.05 | 30 min |
| 31 | | | tertiary butanol | | |
| Formula | KBr | 1.21 | NaCl, 0.1 mM EDTA, 0.5% | 1.05 | 30 min |
| 32 | | | tertiary butanol | | |
| Formula | KBr | 1.21 | NaCl, 0.1 mM EDTA, 1% | 1.05 | 30 min |
| 33 | | | tertiary butanol | | |
| Formula | KBr | 1.21 | NaCl, 0.1 mM EDTA, 5% | 1.03 | 28 min |
| 34 | | | tertiary butanol | | |
| Formula | KBr | 1.21 | NaCl, 0.1 mM EDTA, 25% | 0.98 | 25 min |
| 35 | | | tertiary butanol | | |
| Formula | KBr | 1.21 | NaCl, 0.1 mM EDTA, 50% | 0.95 | 22 min |
| 36 | | | tertiary butanol | | |
| Formula | KBr | 1.21 | NaCl, 0.1 mM EDTA, 0.1% | 1.05 | 30 min |
| 37 | | | ethylene glycol | | |
| Formula | KBr | 1.21 | NaCl, 0.1 mM EDTA, 0.5% | 1.05 | 30 min |
| 38 | | | ethylene glycol | | |
| Formula | KBr | 1.21 | NaCl, 0.1 mM EDTA, 1% | 1.05 | 30 min |
| 39 | | | ethylene glycol | | |
| Formula | KBr | 1.21 | NaCl, 0.1 mM EDTA, 5% | 1.03 | 28 min |
| 40 | | | ethylene glycol | | |
| Formula | KBr | 1.21 | NaCl, 0.1 mM EDTA, 25% | 0.98 | 25 min |
| 41 | | | ethylene glycol | | |
| Formula | KBr | 1.21 | NaCl, 0.1 mM EDTA, 50% | 0.95 | 22 min |
| 42 | | | ethylene glycol | | |
| Formula | KBr | 1.21 | NaCl, 0.1 mM EDTA, 0.1% | 1.05 | 30 min |
| 43 | | | propylene glycol | | |
| Formula | KBr | 1.21 | NaCl, 0.1 mM EDTA, 0.5% | 1.05 | 30 min |
| 44 | | | propylene glycol | | |
| Formula | KBr | 1.21 | NaCl, 0.1 mM EDTA, 1% | 1.05 | 30 min |
| 45 | | | propylene glycol | | |
| Formula | KBr | 1.21 | NaCl, 0.1 mM EDTA, 5% | 1.03 | 28 min |
| 46 | | | propylene glycol | | |
| Formula | KBr | 1.21 | NaCl, 0.1 mM EDTA, 25% | 0.98 | 25 min |
| 47 | | | propylene glycol | | |
| Formula | KBr | 1.21 | NaCl, 0.1 mM EDTA, 50% | 0.95 | 22 min |
| 48 | | | propylene glycol | | |
| Formula | KBr | 1.21 | NaCl, 0.1 mM EDTA, 1% | 1.05 | 30 min |
| 49 | | | methanol | | |
| Formula | NaBr | 1.21 | NaCl, 0.1 mM EDTA, 1% | 1.05 | 30 min |
| 50 | | | ethanol | | |
| Formula | NaBr | 1.21 | NaCl, 0.1 mM EDTA, 1% | 1.05 | 30 min |
| 51 | | | isopropanol | | |
| Formula | NaBr | 1.21 | NaCl, 0.1 mM EDTA, 1% n- | 1.05 | 30 min |
| 52 | | | propanol | | |
| Formula | NaBr | 1.21 | NaCl, 0.1 mM EDTA, 1% n- | 1.05 | 30 min |
| 53 | | | butanol | | |
| Formula | NaBr | 1.21 | NaCl, 0.1 mM EDTA, 1% | 1.05 | 30 min |
| 54 | | | isob uta nol | | |
| Formula | NaBr | 1.21 | NaCl, 0.1 mM EDTA, 1% | 1.05 | 30 min |
| 55 | | | tertiary butanol | | |
| Formula | NaBr | 1.21 | NaCl, 0.1 mM EDTA, 1% | | 1.05 |
| 56 | | | ethylene glycol | | |
| Formula | NaBr | 1.21 | NaCl, 0.1 mM EDTA, 1% | 1.05 | 30 min |
| 57 | | | propylene glycol | | |
| Formula | NaBr | 1.21 | NaCl, 0.1 mM EDTA, 1% | 1.05 | 30 min |
| 58 | | | methanol | | |
| Formula | Sucrose | 1.21 | NaCl, 0.1 mM EDTA, 1% | 1.05 | 30 min |
| 59 | | | ethanol | | |
| Formula | Sucrose | 1.21 | NaCl, 0.1 mM EDTA, 1% | 1.05 | 30 min |
| 60 | | | isopropanol | | |
| Formula | Sucrose | 1.21 | NaCl, 0.1 mM EDTA, 1% n- | 1.05 | 30 min |
| 61 | | | propanol | | |
| Formula | Sucrose | 1.21 | NaCl, 0.1 mM EDTA, 1% n- | 1.05 | 30 min |
| 62 | | | butanol | | |
| Formula | Sucrose | 1.21 | NaCl, 0.1 mM EDTA, 1% | 1.05 | 30 min |
| 63 | | | isobutanol | | |
| Formula | Sucrose | 1.21 | NaCl, 0.1 mM EDTA, 1% | 1.05 | 30 min |
| 64 | | | tertiary butanol | | |
| Formula | Sucrose | 1.21 | NaCl, 0.1 mM EDTA, 1% | 1.05 | 30 min |
| 65 | | | ethylene glycol | | |
| Formula | Sucrose | 1.21 | NaCl, 0.1 mM EDTA, 1% | 1.05 | 30 min |
| 66 | | | propylene glycol | | |
| Formula | Sucrose | 1.21 | NaCl, 0.1 mM EDTA, 1% | 1.05 | 30 min |
| 67 | | | methanol | | |

Some substances in the above Table are not marked with concentrations, for example, KBr is used to adjust the density of the diluent. When the density of the diluent is 1.21, the solid KBr is added until the density is 1.21 g/ml. The same principle is applicable to NaBr and sucrose and the like. NaCl in the components of the density liquid is also used to adjust density. Based on different densities, the addition amount of NaCl will be slightly different.

### Embodiment 2

Precision Test

One sample was taken and detected for 10 times by using the present detection system to calculate the precision of the lipoprotein particle concentration with the result shown in Table 2. The sample formulas used are formulas prepared in the embodiments, for example, the formula 1 in Table 2 corresponds to the formula 1 in Table 1 (components: diluent KBr, density 1.21 g/ml; density liquid NaCl, 0.1 mM EDTA, 0.1% ethanol, density 1.05 g/ml; centrifugation time 30 min) as below.

TABLE 2
Precision (sample 1, TG concentration: 1.5 mM)

| Serial | Repeatability (CV) | | | | |
|---|---|---|---|---|---|
| number | HDL-P | LDL-P | Lpa-P | IDL-P | VLDL-P |
| Control | 3.81% | 2.20% | 5.63% | 4.60% | 4.22% |
| formula | | | | | |
| Formula 1 | 4.24% | 1.64% | 6.52% | 4.94% | 4.90% |
| Formula 2 | 4.78% | 2.74% | 6.01% | 4.10% | 3.31% |
| Formula 3 | 2.88% | 2.54% | 4.48% | 4.96% | 3.34% |
| Formula 4 | 3.57% | 2.19% | 5.64% | 5.85% | 5.44% |
| Formula 5 | 3.00% | 2.10% | 5.12% | 5.69% | 3.68% |
| Formula 6 | 4.27% | 2.84% | 5.95% | 3.24% | 4.58% |
| Formula 7 | 4.12% | 1.82% | 4.68% | 4.75% | 3.99% |
| Formula 8 | 3.45% | 2.28% | 7.27% | 3.81% | 3.15% |
| Formula 9 | 4.38% | 1.69% | 7.03% | 3.71% | 4.69% |
| Formula 10 | 4.82% | 1.84% | 5.33% | 3.81% | 4.22% |
| Formula 11 | 3.28% | 1.97% | 7.21% | 5.50% | 5.16% |
| Formula 12 | 4.60% | 2.02% | 6.81% | 5.06% | 5.18% |
| Formula 13 | 2.82% | 2.27% | 5.06% | 5.20% | 3.98% |
| Formula 14 | 3.98% | 2.39% | 5.78% | 5.54% | 4.79% |
| Formula 15 | 3.77% | 1.79% | 4.27% | 4.07% | 4.69% |
| Formula 16 | 4.65% | 1.75% | 5.02% | 4.43% | 3.00% |
| Formula 17 | 4.09% | 2.56% | 6.21% | 5.53% | 4.14% |
| Formula 18 | 2.83% | 2.46% | 6.10% | 3.43% | 4.67% |
| Formula 19 | 3.69% | 2.27% | 7.01% | 5.37% | 5.29% |
| Formula 20 | 2.69% | 2.65% | 5.20% | 4.85% | 5.01% |
| Formula 21 | 3.95% | 2.20% | 4.35% | 4.36% | 3.54% |
| Formula 22 | 3.20% | 2.21% | 5.51% | 3.83% | 4.69% |
| Formula 23 | 4.08% | 1.55% | 4.27% | 4.39% | 3.70% |
| Formula 24 | 3.08% | 2.61% | 5.86% | 4.22% | 4.37% |
| Formula 25 | 3.40% | 2.77% | 7.08% | 4.95% | 3.87% |
| Formula 26 | 3.09% | 1.70% | 4.92% | 3.79% | 4.99% |
| Formula 27 | 3.83% | 2.71% | 7.18% | 4.72% | 4.58% |
| Formula 28 | 4.39% | 1.89% | 5.80% | 4.44% | 5.16% |
| Formula 29 | 3.87% | 1.80% | 6.32% | 3.70% | 5.30% |
| Formula 30 | 3.97% | 1.60% | 7.09% | 3.46% | 3.69% |
| Formula 31 | 4.73% | 1.88% | 5.42% | 4.30% | 4.43% |
| Formula 32 | 3.97% | 2.85% | 5.78% | 3.30% | 3.13% |
| Formula 33 | 3.58% | 2.24% | 5.59% | 4.45% | 3.58% |
| Formula 34 | 4.09% | 1.75% | 4.18% | 5.49% | 3.57% |
| Formula 35 | 4.14% | 2.19% | 6.93% | 4.01% | 3.58% |
| Formula 36 | 3.63% | 1.61% | 7.37% | 4.87% | 2.97% |
| Formula 37 | 4.82% | 2.00% | 5.94% | 3.87% | 5.14% |
| Formula 38 | 3.29% | 2.18% | 5.25% | 5.28% | 4.42% |
| Formula 39 | 4.02% | 1.77% | 5.10% | 4.45% | 3.11% |
| Formula 40 | 3.83% | 2.64% | 4.32% | 4.94% | 4.77% |
| Formula 41 | 3.97% | 1.89% | 4.32% | 3.88% | 3.55% |
| Formula 42 | 4.57% | 2.30% | 4.54% | 5.08% | 4.69% |
| Formula 43 | 4.84% | 1.83% | 6.12% | 3.75% | 5.19% |
| Formula 44 | 4.20% | 1.84% | 6.94% | 3.83% | 3.30% |
| Formula 45 | 4.94% | 2.65% | 6.91% | 3.31% | 5.22% |
| Formula 46 | 4.28% | 2.45% | 7.30% | 4.74% | 4.36% |
| Formula 47 | 4.23% | 2.26% | 7.35% | 4.27% | 3.33% |
| Formula 48 | 4.10% | 2.75% | 7.26% | 4.71% | 3.53% |
| Formula 49 | 4.09% | 1.59% | 6.82% | 4.27% | 4.31% |
| Formula 50 | 3.20% | 2.06% | 7.40% | 5.61% | 4.76% |
| Formula 51 | 4.25% | 2.55% | 5.75% | 3.86% | 4.28% |
| Formula 52 | 3.10% | 2.28% | 5.36% | 4.40% | 2.99% |
| Formula 53 | 4.81% | 2.84% | 7.25% | 3.35% | 4.61% |
| Formula 54 | 2.93% | 2.54% | 7.33% | 5.17% | 3.85% |
| Formula 55 | 4.87% | 2.31% | 5.04% | 3.73% | 5.41% |
| Formula 56 | 4.12% | 1.79% | 6.60% | 5.20% | 5.43% |
| Formula 57 | 3.78% | 2.31% | 6.80% | 5.17% | 3.80% |
| Formula 58 | 3.77% | 1.76% | 4.83% | 5.47% | 3.60% |
| Formula 59 | 4.60% | 2.61% | 5.06% | 4.25% | 4.44% |
| Formula 60 | 3.38% | 1.63% | 7.17% | 5.71% | 4.75% |
| Formula 61 | 3.20% | 1.87% | 6.60% | 5.76% | 3.41% |
| Formula 62 | 2.83% | 1.84% | 4.27% | 3.56% | 3.67% |
| Formula 63 | 3.41% | 2.41% | 7.23% | 5.06% | 5.40% |
| Formula 64 | 3.62% | 1.55% | 6.30% | 3.60% | 3.89% |
| Formula 65 | 4.89% | 2.13% | 6.24% | 4.55% | 3.51% |
| Formula 66 | 3.62% | 2.82% | 6.09% | 4.02% | 3.32% |
| Formula 67 | 4.53% | 1.80% | 4.75% | 5.82% | 4.14% |

TABLE 3
Precision (sample 2, TG concentration: 2.0 mM)

| Serial | Repeatability (CV) | | | | |
|---|---|---|---|---|---|
| number | HDL-P | LDL-P | Lpa-P | IDL-P | VLDL-P |
| Control | 4.86% | 3.20% | 7.82% | 5.96% | 5.62% |
| Formula | | | | | |
| Formula 1 | 3.06% | 2.47% | 4.13% | 3.77% | 4.22% |
| Formula 2 | 4.72% | 2.55% | 5.11% | 4.06% | 3.36% |
| Formula 3 | 4.13% | 2.36% | 6.96% | 3.31% | 4.60% |
| Formula 4 | 3.77% | 1.80% | 4.25% | 4.93% | 4.73% |
| Formula 5 | 2.88% | 2.63% | 5.52% | 3.30% | 4.28% |
| Formula 6 | 4.14% | 2.58% | 4.97% | 4.03% | 4.60% |
| Formula 7 | 3.92% | 2.39% | 4.35% | 5.76% | 3.34% |
| Formula 8 | 4.07% | 2.45% | 5.29% | 3.51% | 4.19% |
| Formula 9 | 2.93% | 1.73% | 6.27% | 5.31% | 3.46% |
| Formula 10 | 4.06% | 1.66% | 4.51% | 5.38% | 5.45% |
| Formula 11 | 3.01% | 2.68% | 5.65% | 3.88% | 4.65% |
| Formula 12 | 4.67% | 1.64% | 6.24% | 3.31% | 3.26% |
| Formula 13 | 4.86% | 2.62% | 7.34% | 3.92% | 4.63% |
| Formula 14 | 3.27% | 1.82% | 6.36% | 5.15% | 4.84% |
| Formula 15 | 4.66% | 1.87% | 6.25% | 4.57% | 4.84% |
| Formula 16 | 2.85% | 1.89% | 6.75% | 5.59% | 5.36% |
| Formula 17 | 4.27% | 2.36% | 6.74% | 5.36% | 4.46% |
| Formula 18 | 4.84% | 2.45% | 4.38% | 5.84% | 3.44% |
| Formula 19 | 3.74% | 1.83% | 5.57% | 5.38% | 4.42% |
| Formula 20 | 3.00% | 2.68% | 4.32% | 4.82% | 3.57% |
| Formula 21 | 3.49% | 2.73% | 5.70% | 3.68% | 5.11% |
| Formula 22 | 3.94% | 1.70% | 6.54% | 4.89% | 4.16% |
| Formula 23 | 3.22% | 2.50% | 7.30% | 4.32% | 5.16% |
| Formula 24 | 4.23% | 2.45% | 6.70% | 4.30% | 3.02% |
| Formula 25 | 3.27% | 2.59% | 6.46% | 5.83% | 3.97% |
| Formula 26 | 3.34% | 1.90% | 4.97% | 5.80% | 4.75% |
| Formula 27 | 4.09% | 1.64% | 6.53% | 4.78% | 4.34% |
| Formula 28 | 3.46% | 2.53% | 6.03% | 3.48% | 4.49% |
| Formula 29 | 3.63% | 1.83% | 6.69% | 4.76% | 3.77% |
| Formula 30 | 4.67% | 2.11% | 4.52% | 3.70% | 4.62% |
| Formula 31 | 3.00% | 2.37% | 6.93% | 4.95% | 4.41% |
| Formula 32 | 2.71% | 2.66% | 4.14% | 3.51% | 4.07% |
| Formula 33 | 3.89% | 2.13% | 4.71% | 5.36% | 5.14% |
| Formula 34 | 3.79% | 1.59% | 7.23% | 3.54% | 4.17% |
| Formula 35 | 2.76% | 2.51% | 6.27% | 4.66% | 4.15% |
| Formula 36 | 3.12% | 1.77% | 6.85% | 4.60% | 3.35% |
| Formula 37 | 4.73% | 1.57% | 4.98% | 5.85% | 5.13% |
| Formula 38 | 2.72% | 1.84% | 6.77% | 3.98% | 3.40% |
| Formula 39 | 3.87% | 1.81% | 5.34% | 3.98% | 3.39% |
| Formula 40 | 3.72% | 1.66% | 4.00% | 3.57% | 3.78% |
| Formula 41 | 3.05% | 1.73% | 6.22% | 4.66% | 5.45% |
| Formula 42 | 3.04% | 2.48% | 5.52% | 5.15% | 4.47% |
| Formula 43 | 4.00% | 2.07% | 5.10% | 4.54% | 3.72% |
| Formula 44 | 3.63% | 1.64% | 4.08% | 3.47% | 4.58% |
| Formula 45 | 3.22% | 1.61% | 5.41% | 4.68% | 4.47% |
| Formula 46 | 4.31% | 2.10% | 5.81% | 3.88% | 5.34% |
| Formula 47 | 3.29% | 1.98% | 6.88% | 3.59% | 2.97% |
| Formula 48 | 4.22% | 2.62% | 6.11% | 4.65% | 5.18% |
| Formula 49 | 3.56% | 2.69% | 5.10% | 3.43% | 4.67% |
| Formula 50 | 4.74% | 2.72% | 5.57% | 3.61% | 3.09% |
| Formula 51 | 4.91% | 2.34% | 4.50% | 4.39% | 4.63% |
| Formula 52 | 4.39% | 2.59% | 6.17% | 5.84% | 5.16% |
| Formula 53 | 2.77% | 1.94% | 7.38% | 5.33% | 4.45% |
| Formula 54 | 3.26% | 1.75% | 4.33% | 4.93% | 3.29% |
| Formula 55 | 3.14% | 2.79% | 6.90% | 5.88% | 4.77% |
| Formula 56 | 3.04% | 1.75% | 7.02% | 3.94% | 4.11% |
| Formula 57 | 3.10% | 1.55% | 4.96% | 3.91% | 4.20% |
| Formula 58 | 4.36% | 2.55% | 6.52% | 4.46% | 4.99% |
| Formula 59 | 4.18% | 2.84% | 5.52% | 5.23% | 4.07% |
| Formula 60 | 4.08% | 1.59% | 6.67% | 5.84% | 5.09% |
| Formula 61 | 4.33% | 2.80% | 4.78% | 4.90% | 3.03% |
| Formula 62 | 3.79% | 2.80% | 5.82% | 5.73% | 2.98% |
| Formula 63 | 3.74% | 2.33% | 4.83% | 4.89% | 5.22% |
| Formula 64 | 3.60% | 2.62% | 6.34% | 4.11% | 4.47% |
| Formula 65 | 4.15% | 2.49% | 6.50% | 3.52% | 5.03% |
| Formula 66 | 4.02% | 2.72% | 6.84% | 5.81% | 3.02% |
| Formula 67 | 3.78% | 2.72% | 4.54% | 5.28% | 3.92% |

TABLE 4
Precision (sample 3, TG concentration: 3.2 mM)

| Serial | Repeatability (CV) | | | | |
|---|---|---|---|---|---|
| number | HDL-P | LDL-P | Lpa-P | IDL-P | VLDL-P |
| Control | 7.12% | 5.20% | 8.86% | 9.26% | 6.23% |
| Formula | | | | | |
| Formula 1 | 2.74% | 2.44% | 5.59% | 3.96% | 3.12% |
| Formula 2 | 4.52% | 1.64% | 4.61% | 4.96% | 3.91% |
| Formula 3 | 2.70% | 2.68% | 5.83% | 3.40% | 4.59% |
| Formula 4 | 3.01% | 1.59% | 6.85% | 3.24% | 4.18% |
| Formula 5 | 2.79% | 1.71% | 4.55% | 3.76% | 3.49% |
| Formula 6 | 3.25% | 2.38% | 5.74% | 5.11% | 4.40% |
| Formula 7 | 4.31% | 2.23% | 6.41% | 3.82% | 5.28% |
| Formula 8 | 3.00% | 2.56% | 7.34% | 4.92% | 3.79% |
| Formula 9 | 3.45% | 2.54% | 5.37% | 3.32% | 5.11% |
| Formula 10 | 3.58% | 2.74% | 7.27% | 5.54% | 5.29% |
| Formula 11 | 2.81% | 1.72% | 7.26% | 3.71% | 3.94% |
| Formula 12 | 3.86% | 1.94% | 7.14% | 3.56% | 3.31% |
| Formula 13 | 3.64% | 2.14% | 4.24% | 3.62% | 3.06% |
| Formula 14 | 3.81% | 2.77% | 6.97% | 3.82% | 3.05% |
| Formula 15 | 3.90% | 2.75% | 5.83% | 3.97% | 5.31% |
| Formula 16 | 3.92% | 2.53% | 4.85% | 3.52% | 3.53% |
| Formula 17 | 4.70% | 2.40% | 4.80% | 5.28% | 5.38% |
| Formula 18 | 2.97% | 2.67% | 6.67% | 5.04% | 4.40% |
| Formula 19 | 4.68% | 2.41% | 5.15% | 4.05% | 5.08% |
| Formula 20 | 4.82% | 1.62% | 5.95% | 5.11% | 3.24% |
| Formula 21 | 3.67% | 2.69% | 6.67% | 5.83% | 4.71% |
| Formula 22 | 4.77% | 1.58% | 5.40% | 4.54% | 4.02% |
| Formula 23 | 3.80% | 2.05% | 4.38% | 3.65% | 2.95% |
| Formula 24 | 2.91% | 1.86% | 6.78% | 5.13% | 5.04% |
| Formula 25 | 4.81% | 2.63% | 7.41% | 3.40% | 5.24% |
| Formula 26 | 3.86% | 2.56% | 5.35% | 5.04% | 4.26% |
| Formula 27 | 2.92% | 2.71% | 6.02% | 4.47% | 4.51% |
| Formula 28 | 2.93% | 1.68% | 6.94% | 5.23% | 5.17% |
| Formula 29 | 3.18% | 2.86% | 4.88% | 3.77% | 4.36% |
| Formula 30 | 4.62% | 2.80% | 6.02% | 5.20% | 4.83% |
| Formula 31 | 4.53% | 2.49% | 5.80% | 3.37% | 5.32% |
| Formula 32 | 3.82% | 2.32% | 5.20% | 4.60% | 3.81% |
| Formula 33 | 2.70% | 1.75% | 7.33% | 3.78% | 5.42% |
| Formula 34 | 4.89% | 1.97% | 4.14% | 5.42% | 3.00% |
| Formula 35 | 4.46% | 1.87% | 5.55% | 5.17% | 4.62% |
| Formula 36 | 4.67% | 1.55% | 6.06% | 4.05% | 4.40% |
| Formula 37 | 3.36% | 2.75% | 7.26% | 4.95% | 3.79% |
| Formula 38 | 4.14% | 2.11% | 7.25% | 3.98% | 3.47% |
| Formula 39 | 2.66% | 2.54% | 6.73% | 3.23% | 3.98% |
| Formula 40 | 4.92% | 2.18% | 5.65% | 4.57% | 5.08% |
| Formula 41 | 3.43% | 2.30% | 7.06% | 5.30% | 3.91% |
| Formula 42 | 4.70% | 2.27% | 5.85% | 3.79% | 4.85% |
| Formula 43 | 4.80% | 1.98% | 4.49% | 5.96% | 3.54% |
| Formula 44 | 3.03% | 2.22% | 4.39% | 4.65% | 5.08% |
| Formula 45 | 3.17% | 2.48% | 6.37% | 3.68% | 5.43% |
| Formula 46 | 3.89% | 1.61% | 4.36% | 4.40% | 4.22% |
| Formula 47 | 4.39% | 1.90% | 4.03% | 4.01% | 5.33% |
| Formula 48 | 3.73% | 2.23% | 5.24% | 5.73% | 3.49% |
| Formula 49 | 4.34% | 2.74% | 5.63% | 3.91% | 4.61% |
| Formula 50 | 4.20% | 1.59% | 5.91% | 3.88% | 4.55% |
| Formula 51 | 2.96% | 2.17% | 6.59% | 5.75% | 4.49% |
| Formula 52 | 4.36% | 2.49% | 6.41% | 4.51% | 3.39% |
| Formula 53 | 3.35% | 2.24% | 7.20% | 3.27% | 4.24% |
| Formula 54 | 3.39% | 2.33% | 6.55% | 4.42% | 3.98% |
| Formula 55 | 3.10% | 2.76% | 7.18% | 3.98% | 4.60% |
| Formula 56 | 3.76% | 2.17% | 6.76% | 3.54% | 3.96% |
| Formula 57 | 3.76% | 1.65% | 5.47% | 5.62% | 3.26% |
| Formula 58 | 4.60% | 2.39% | 4.66% | 4.02% | 3.35% |
| Formula 59 | 3.44% | 2.08% | 5.87% | 5.70% | 3.05% |
| Formula 60 | 4.86% | 2.35% | 4.44% | 5.48% | 3.12% |
| Formula 61 | 3.79% | 2.41% | 5.98% | 3.22% | 4.88% |
| Formula 62 | 4.77% | 2.30% | 7.26% | 5.74% | 5.35% |
| Formula 63 | 3.66% | 2.82% | 7.02% | 4.68% | 4.55% |
| Formula 64 | 4.06% | 2.30% | 5.52% | 5.04% | 5.32% |
| Formula 65 | 4.39% | 2.38% | 7.16% | 3.71% | 4.22% |
| Formula 66 | 4.49% | 2.56% | 4.60% | 5.76% | 4.28% |
| Formula 67 | 2.69% | 2.02% | 5.55% | 3.86% | 3.06% |

TABLE 5
Precision (Sample 4, TG concentration: 4.5 mM)

| Serial | Repeatability (CV) | | | | |
|---|---|---|---|---|---|
| number | HDL-P | LDL-P | Lpa-P | IDL-P | VLDL-P |
| Control | 10.96% | 9.20% | 14.70% | 13.60% | 12.20% |
| Formula | | | | | |
| Formula 1 | 4.51% | 1.58% | 5.47% | 4.18% | 3.14% |
| Formula 2 | 2.95% | 1.59% | 4.08% | 5.71% | 3.12% |
| Formula 3 | 3.61% | 2.22% | 5.19% | 3.94% | 4.55% |
| Formula 4 | 3.02% | 2.74% | 5.16% | 5.02% | 4.90% |
| Formula 5 | 2.86% | 2.29% | 6.13% | 5.85% | 3.83% |
| Formula 6 | 4.92% | 2.02% | 4.37% | 4.92% | 3.14% |
| Formula 7 | 4.32% | 2.17% | 5.88% | 4.60% | 5.05% |
| Formula 8 | 2.71% | 2.17% | 4.67% | 4.53% | 5.18% |
| Formula 9 | 2.95% | 2.50% | 6.46% | 5.19% | 5.46% |
| Formula 10 | 4.47% | 2.37% | 5.03% | 3.75% | 4.55% |
| Formula 11 | 4.54% | 1.86% | 5.99% | 4.05% | 5.43% |
| Formula 12 | 4.72% | 2.70% | 5.36% | 5.40% | 4.28% |
| Formula 13 | 4.09% | 2.13% | 6.21% | 4.53% | 4.75% |
| Formula 14 | 4.36% | 2.05% | 6.98% | 4.56% | 3.80% |
| Formula 15 | 3.71% | 2.63% | 5.09% | 5.42% | 4.13% |
| Formula 16 | 4.17% | 2.59% | 6.84% | 3.43% | 4.91% |
| Formula 17 | 3.92% | 2.55% | 6.54% | 5.43% | 3.58% |
| Formula 18 | 3.03% | 2.57% | 4.68% | 3.79% | 3.98% |
| Formula 19 | 3.23% | 2.37% | 4.00% | 3.95% | 3.19% |
| Formula 20 | 4.33% | 1.56% | 5.81% | 3.32% | 5.00% |
| Formula 21 | 2.66% | 1.86% | 4.81% | 3.37% | 4.82% |
| Formula 22 | 3.08% | 1.62% | 6.07% | 5.94% | 4.88% |
| Formula 23 | 2.95% | 2.52% | 6.78% | 5.02% | 4.32% |
| Formula 24 | 4.61% | 2.84% | 5.38% | 4.31% | 3.01% |
| Formula 25 | 4.46% | 2.81% | 6.63% | 4.07% | 3.18% |
| Formula 26 | 3.48% | 1.63% | 5.00% | 3.30% | 4.43% |
| Formula 27 | 3.78% | 1.74% | 6.78% | 5.40% | 3.91% |
| Formula 28 | 4.81% | 1.65% | 5.05% | 3.86% | 3.27% |
| Formula 29 | 4.26% | 1.70% | 6.19% | 4.75% | 4.39% |
| Formula 30 | 3.10% | 2.65% | 4.72% | 4.66% | 3.08% |
| Formula 31 | 2.80% | 1.89% | 6.32% | 5.59% | 4.71% |
| Formula 32 | 3.20% | 2.79% | 5.21% | 3.69% | 3.20% |
| Formula 33 | 3.75% | 2.80% | 5.15% | 5.85% | 4.79% |
| Formula 34 | 4.05% | 2.03% | 6.15% | 5.14% | 3.88% |
| Formula 35 | 2.94% | 2.24% | 4.66% | 4.03% | 4.27% |
| Formula 36 | 4.91% | 2.83% | 4.06% | 5.41% | 3.82% |
| Formula 37 | 4.85% | 1.68% | 5.58% | 5.54% | 4.00% |
| Formula 38 | 3.78% | 1.62% | 5.58% | 5.02% | 5.11% |
| Formula 39 | 3.86% | 1.62% | 7.03% | 4.85% | 5.33% |
| Formula 40 | 4.76% | 1.71% | 4.35% | 3.69% | 4.09% |
| Formula 41 | 3.81% | 2.41% | 5.47% | 5.84% | 3.70% |
| Formula 42 | 3.70% | 1.56% | 5.03% | 5.31% | 3.12% |
| Formula 43 | 4.90% | 2.04% | 6.43% | 3.56% | 3.11% |
| Formula 44 | 3.56% | 2.50% | 4.51% | 3.23% | 3.92% |
| Formula 45 | 2.99% | 2.14% | 6.55% | 4.26% | 5.24% |
| Formula 46 | 4.42% | 2.55% | 6.47% | 4.75% | 3.47% |
| Formula 47 | 4.11% | 1.98% | 4.12% | 4.83% | 3.91% |
| Formula 48 | 4.78% | 2.20% | 4.06% | 5.46% | 3.84% |
| Formula 49 | 3.84% | 1.73% | 6.22% | 4.70% | 4.31% |
| Formula 50 | 4.31% | 2.12% | 5.95% | 4.82% | 4.11% |
| Formula 51 | 3.07% | 2.55% | 5.11% | 5.55% | 5.07% |
| Formula 52 | 4.34% | 2.01% | 5.58% | 5.79% | 5.05% |
| Formula 53 | 4.01% | 2.77% | 5.52% | 3.59% | 5.10% |
| Formula 54 | 4.05% | 2.04% | 5.09% | 5.43% | 3.08% |
| Formula 55 | 3.47% | 2.08% | 6.14% | 3.73% | 5.06% |
| Formula 56 | 4.69% | 2.44% | 6.01% | 5.19% | 4.46% |
| Formula 57 | 4.15% | 1.76% | 6.00% | 4.95% | 4.37% |
| Formula 58 | 3.58% | 2.66% | 6.14% | 4.24% | 5.03% |
| Formula 59 | 3.19% | 2.39% | 5.14% | 4.72% | 3.58% |
| Formula 60 | 4.51% | 2.23% | 4.99% | 4.18% | 3.54% |
| Formula 61 | 3.80% | 2.42% | 6.91% | 3.91% | 4.28% |
| Formula 62 | 2.69% | 2.81% | 5.42% | 4.62% | 4.26% |
| Formula 63 | 2.73% | 2.84% | 6.08% | 4.94% | 4.49% |
| Formula 64 | 3.46% | 2.10% | 5.64% | 5.89% | 3.68% |
| Formula 65 | 4.41% | 1.54% | 6.15% | 5.26% | 4.93% |
| Formula 66 | 4.47% | 1.72% | 5.75% | 3.28% | 4.06% |
| Formula 67 | 4.30% | 2.55% | 5.20% | 3.34% | 4.31% |

TABLE 6
Precision (Sample 5, TG concentration: 6.2 mM)

| Serial | Repeatability (CV) | | | | |
|---|---|---|---|---|---|
| number | HDL-P | LDL-P | Lpa-P | IDL-P | VLDL-P |
| Control | 16.36% | 10.23% | 22.70% | 19.20% | 18.20% |
| Formula | | | | | |
| Formula 1 | 3.25% | 2.37% | 6.83% | 5.66% | 4.49% |
| Formula 2 | 3.13% | 1.79% | 7.22% | 4.82% | 4.90% |
| Formula 3 | 3.96% | 2.64% | 5.87% | 5.33% | 4.13% |
| Formula 4 | 4.71% | 2.72% | 5.73% | 4.06% | 3.86% |
| Formula 5 | 4.59% | 2.48% | 5.76% | 3.52% | 4.79% |
| Formula 6 | 3.23% | 2.84% | 6.30% | 4.44% | 4.73% |
| Formula 7 | 2.69% | 2.47% | 4.90% | 5.55% | 3.71% |
| Formula 8 | 4.01% | 1.72% | 5.05% | 3.81% | 3.51% |
| Formula 9 | 4.44% | 2.72% | 7.31% | 4.55% | 5.41% |
| Formula 10 | 4.06% | 2.49% | 5.75% | 4.48% | 4.56% |
| Formula 11 | 4.85% | 2.26% | 4.68% | 4.86% | 3.33% |
| Formula 12 | 3.71% | 2.24% | 6.58% | 3.23% | 3.72% |
| Formula 13 | 4.45% | 2.62% | 6.51% | 4.62% | 3.52% |
| Formula 14 | 4.23% | 2.34% | 6.01% | 4.37% | 4.33% |
| Formula 15 | 4.93% | 1.55% | 4.00% | 4.88% | 4.60% |
| Formula 16 | 4.41% | 1.63% | 5.53% | 5.38% | 5.35% |
| Formula 17 | 2.74% | 2.53% | 5.77% | 3.27% | 3.61% |
| Formula 18 | 4.47% | 2.30% | 6.00% | 4.22% | 4.96% |
| Formula 19 | 4.67% | 1.93% | 4.45% | 4.49% | 3.27% |
| Formula 20 | 3.71% | 1.76% | 7.37% | 4.66% | 3.00% |
| Formula 21 | 3.65% | 1.57% | 7.17% | 3.79% | 3.29% |
| Formula 22 | 3.88% | 1.93% | 4.83% | 4.76% | 3.21% |
| Formula 23 | 4.21% | 1.84% | 5.13% | 5.85% | 5.32% |
| Formula 24 | 4.88% | 2.23% | 6.94% | 4.72% | 4.23% |
| Formula 25 | 3.88% | 2.68% | 6.21% | 4.34% | 4.30% |
| Formula 26 | 4.93% | 2.27% | 4.35% | 4.83% | 5.05% |
| Formula 27 | 4.41% | 2.01% | 4.68% | 4.60% | 4.62% |
| Formula 28 | 4.17% | 1 2.18% | 7.07% | 5.59% | 4.99% |
| Formula 29 | 4.28% | 2.33% | 4.20% | 4.54% | 5.21% |
| Formula 30 | 3.39% | 2.72% | 7.13% | 5.67% | 4.41% |
| Formula 31 | 3.01% | 1.81% | 4.64% | 5.90% | 5.35% |
| Formula 32 | 2.85% | 2.84% | 4.90% | 5.21% | 4.41% |
| Formula 33 | 4.61% | 2.00% | 6.11% | 5.70% | 4.39% |
| Formula 34 | 4.12% | 1.99% | 5.26% | 3.33% | 3.31% |
| Formula 35 | 3.61% | 2.29% | 6.08% | 5.59% | 5.12% |
| Formula 36 | 4.40% | 2.69% | 4.99% | 3.53% | 3.50% |
| Formula 37 | 3.77% | 2.78% | 7.13% | 3.89% | 3.86% |
| Formula 38 | 4.31% | 2.75% | 5.59% | 5.36% | 3.61% |
| Formula 39 | 3.81% | 1.81% | 6.74% | 5.44% | 4.60% |
| Formula 40 | 3.79% | 1.80% | 6.91% | 5.00% | 4.94% |
| Formula 41 | 4.05% | 2.82% | 4.18% | 5.38% | 3.08% |
| Formula 42 | 4.47% | 2.51% | 6.46% | 4.25% | 3.00% |
| Formula 43 | 4.56% | 2.02% | 5.98% | 5.10% | 5.00% |
| Formula 44 | 4.47% | 2.71% | 4.51% | 3.67% | 5.16% |
| Formula 45 | 3.08% | 2.24% | 6.87% | 5.52% | 4.25% |
| Formula 46 | 4.51% | 2.53% | 4.80% | 3.36% | 4.52% |
| Formula 47 | 4.13% | 2.07% | 5.49% | 3.65% | 3.10% |
| Formula 48 | 4.60% | 1.74% | 4.51% | 3.58% | 4.29% |
| Formula 49 | 4.42% | 1.57% | 5.63% | 5.27% | 3.91% |
| Formula 50 | 4.61% | 1.63% | 4.34% | 5.44% | 3.41% |
| Formula 51 | 3.61% | 1.88% | 4.37% | 5.39% | 4.37% |
| Formula 52 | 3.31% | 1.66% | 5.74% | 4.78% | 3.92% |
| Formula 53 | 3.15% | 2.58% | 5.10% | 4.60% | 4.74% |
| Formula 54 | 3.65% | 1.58% | 5.50% | 5.25% | 3.28% |
| Formula 55 | 4.11% | 2.69% | 7.11% | 3.87% | 4.96% |
| Formula 56 | 3.62% | 1.55% | 4.74% | 5.41% | 5.21% |
| Formula 57 | 4.68% | 2.35% | 4.42% | 5.43% | 3.32% |
| Formula 58 | 4.10% | 2.12% | 7.28% | 5.25% | 3.57% |
| Formula 59 | 2.88% | 1.92% | 4.30% | 3.65% | 5.37% |
| Formula 60 | 3.80% | 2.81% | 6.21% | 4.28% | 4.71% |
| Formula 61 | 2.97% | 2.42% | 6.95% | 3.94% | 5.22% |
| Formula 62 | 2.96% | 2.62% | 7.08% | 5.96% | 3.46% |
| Formula 63 | 3.73% | 1.89% | 5.86% | 5.34% | 3.15% |
| Formula 64 | 4.25% | 2.58% | 4.78% | 3.68% | 3.85% |
| Formula 65 | 3.70% | 1.99% | 7.00% | 5.77% | 4.31% |
| Formula 66 | 4.62% | 2.64% | 4.58% | 5.37% | 4.35% |
| Formula 67 | 4.37% | 2.59% | 4.17% | 3.67% | 4.27% |

It can be seen that the present detection system can detect several different lipoprotein particle concentrations with good precision. In comparison, the precision of the control group enables CV to increase along with increase of the triglyceride concentration in the sample. When the TG concentration in the sample is 2 mM, it is found that the CV is slightly deteriorated; when the TG concentration in the sample is greater than 3.2 mM, it can be seen that the precisions of the several items change much when the TG concentration in the sample is greater than 4.5 mM, the CV is basically above 10%. When the TG concentration continues increasing, the CV will be further deteriorated. The precision of the reagent of the present invention has no sign of deterioration. Thus, the present invention has good clinical application prospect.

### Embodiment 3

Linearity Test

Each item has a high value sample which is diluted to different concentration gradients by using water and then detected using the present detection system to verify its linearity range. The specific linearity ranges of the verified items are HDL-P: 1-80 umol/L; LDL-P: 20-6000 nmol/L; Lpa-P: 2-300 nmol/L; IDL-P:1-100 nmol/L; VLDL-P:2-500 nmo/L. The specific verification results R2 of the linearity ranges are shown in Table 7.

TABLE 7
Verification results of linearity ranges

| Serial | Linearity (R²) | | | | |
|---|---|---|---|---|---|
| number | HDL-P | LDL-P | Lpa-P | IDL-P | VLDL-P |
| Control | 0.9915 | 0.9969 | 0.9890 | 0.9893 | 0.9896 |
| Formula | | | | | |
| Formula 1 | 0.9907 | 0.9936 | 0.9883 | 0.9837 | 0.9866 |
| Formula 2 | 0.9898 | 0.9956 | 0.9857 | 0.9917 | 0.9920 |
| Formula 3 | 0.9902 | 0.9916 | 0.9830 | 0.9876 | 0.9913 |
| Formula 4 | 0.9915 | 0.9984 | 0.9881 | 0.9909 | 0.9878 |
| Formula 5 | 0.9951 | 0.9979 | 0.9844 | 0.9879 | 0.9853 |
| Formula 6 | 0.9916 | 0.9963 | 0.9839 | 0.9923 | 0.9851 |
| Formula 7 | 0.9950 | 0.9940 | 0.9873 | 0.9925 | 0.9879 |
| Formula 8 | 0.9862 | 0.9925 | 0.9908 | 0.9847 | 0.9882 |
| Formula 9 | 0.9925 | 0.9915 | 0.9844 | 0.9838 | 0.9902 |
| Formula 10 | 0.9922 | 0.9956 | 0.9845 | 0.9872 | 0.9918 |
| Formula 11 | 0.9885 | 0.9922 | 0.9847 | 0.9842 | 0.9892 |
| Formula 12 | 0.9916 | 0.9979 | 0.9838 | 0.9915 | 0.9873 |
| Formula 13 | 0.9900 | 0.9974 | 0.9903 | 0.9865 | 0.9907 |
| Formula 14 | 0.9869 | 0.9902 | 0.9915 | 0.9864 | 0.9918 |
| Formula 15 | 0.9921 | 0.9910 | 0.9857 | 0.9900 | 0.9905 |
| Formula 16 | 0.9912 | 0.9915 | 0.9817 | 0.9878 | 0.9885 |
| Formula 17 | 0.9887 | 0.9910 | 0.9840 | 0.9914 | 0.9910 |
| Formula 18 | 0.9870 | 0.9908 | 0.9892 | 0.9890 | 0.9930 |
| Formula 19 | 0.9901 | 0.9921 | 0.9819 | 0.9900 | 0.9902 |
| Formula 20 | 0.9878 | 0.9960 | 0.9820 | 0.9911 | 0.9932 |
| Formula 21 | 0.9876 | 0.9943 | 0.9834 | 0.9864 | 0.9847 |
| Formula 22 | 0.9954 | 0.9992 | 0.9887 | 0.9852 | 0.9930 |
| Formula 23 | 0.9956 | 0.9946 | 0.9837 | 0.9903 | 0.9859 |
| Formula 24 | 0.9890 | 0.9930 | 0.9865 | 0.9855 | 0.9897 |
| Formula 25 | 0.9910 | 0.9938 | 0.9915 | 0.9878 | 0.9927 |
| Formula 26 | 0.9906 | 0.9968 | 0.9831 | 0.9894 | 0.9890 |
| Formula 27 | 0.9933 | 0.9937 | 0.9841 | 0.9874 | 0.9846 |
| Formula 28 | 0.9912 | 0.9969 | 0.9913 | 0.9836 | 0.9930 |
| Formula 29 | 0.9891 | 0.9918 | 0.9877 | 0.9861 | 0.9904 |
| Formula 30 | 0.9881 | 0.9972 | 0.9889 | 0.9884 | 0.9896 |
| Formula 31 | 0.9941 | 0.9979 | 0.9845 | 0.9924 | 0.9933 |
| Formula 32 | 0.9908 | 0.9917 | 0.9895 | 0.9924 | 0.9846 |
| Formula 33 | 0.9918 | 0.9997 | 0.9853 | 0.9849 | 0.9899 |
| Formula 34 | 0.9891 | 0.9936 | 0.9838 | 0.9894 | 0.9853 |
| Formula 35 | 0.9878 | 0.9929 | 0.9877 | 0.9920 | 0.9931 |
| Formula 36 | 0.9912 | 0.9930 | 0.9895 | 0.9911 | 0.9866 |
| Formula 37 | 0.9882 | 0.9982 | 0.9817 | 0.9872 | 0.9931 |
| Formula 38 | 0.9904 | 0.9964 | 0.9823 | 0.9898 | 0.9888 |
| Formula 39 | 0.9878 | 0.9963 | 0.9882 | 0.9914 | 0.9907 |
| Formula 40 | 0.9874 | 0.9905 | 0.9842 | 0.9887 | 0.9890 |
| Formula 41 | 0.9901 | 0.9974 | 0.9864 | 0.9849 | 0.9877 |
| Formula 42 | 0.9919 | 0.9924 | 0.9901 | 0.9881 | 0.9884 |
| Formula 43 | 0.9889 | 0.9915 | 0.9869 | 0.9883 | 0.9871 |
| Formula 44 | 0.9945 | 0.9991 | 0.9905 | 0.9904 | 0.9926 |
| Formula 45 | 0.9956 | 0.9921 | 0.9885 | 0.9894 | 0.9898 |
| Formula 46 | 0.9930 | 0.9906 | 0.9905 | 0.9867 | 0.9868 |
| Formula 47 | 0.9890 | 0.9940 | 0.9877 | 0.9919 | 0.9905 |
| Formula 48 | 0.9958 | 0.9933 | 0.9887 | 0.9856 | 0.9874 |
| Formula 49 | 0.9901 | 0.9972 | 0.9832 | 0.9874 | 0.9892 |
| Formula 50 | 0.9870 | 0.9964 | 0.9832 | 0.9892 | 0.9923 |
| Formula 51 | 0.9890 | 0.9963 | 0.9901 | 0.9834 | 0.9872 |
| Formula 52 | 0.9894 | 0.9994 | 0.9847 | 0.9928 | 0.9853 |
| Formula 53 | 0.9920 | 0.9924 | 0.9859 | 0.9832 | 0.9866 |
| Formula 54 | 0.9932 | 0.9976 | 0.9872 | 0.9905 | 0.9892 |
| Formula 55 | 0.9905 | 0.9986 | 0.9897 | 0.9892 | 0.9933 |
| Formula 56 | 0.9867 | 0.9992 | 0.9855 | 0.9907 | 0.9896 |
| Formula 57 | 0.9916 | 0.9932 | 0.9868 | 0.9867 | 0.9840 |
| Formula 58 | 0.9954 | 0.9941 | 0.9824 | 0.9911 | 0.9874 |
| Formula 59 | 0.9866 | 0.9976 | 0.9890 | 0.9849 | 0.9854 |
| Formula 60 | 0.9873 | 0.9929 | 0.9832 | 0.9875 | 0.9879 |
| Formula 61 | 0.9878 | 0.9985 | 0.9874 | 0.9882 | 0.9836 |
| Formula 62 | 0.9893 | 0.9981 | 0.9913 | 0.9905 | 0.9923 |
| Formula 63 | 0.9920 | 0.9938 | 0.9823 | 0.9913 | 0.9852 |
| Formula 64 | 0.9920 | 0.9956 | 0.9877 | 0.9832 | 0.9854 |
| Formula 65 | 0.9923 | 0.9971 | 0.9880 | 0.9854 | 0.9886 |
| Formula 66 | 0.9939 | 0.9934 | 0.9844 | 0.9891 | 0.9906 |
| Formula 67 | 0.9866 | 0.9991 | 0.9871 | 0.9871 | 0.9837 |

It can be seen that the linearity ranges of several items of lipoprotein particle concentration can satisfy the index requirements and cover the scope required for clinical detection and thus it has good linearity within the linearity range.

### Embodiment 4

Interference Test

Two parts of mixed serum were prepared. One part of mixed serum was mixed with a normal triglyceride sample and its TG concentration was detected to be 1.2 mM; the other part was mixed with a high triglyceride sample and its TG concentration was detected to be 4.2 mM. Mixed based on equal proportion, it has a TG concentration of 2.7 mM. Given a condition, its interference effect is calculated as follows: (high TG sample measured value+low TG sample measured value)/(measured value of equal-proportion-mixed sample*2). It is noted that the high TG sample has much smaller interference capability after being diluted two times by the low TG sample. In fact, there is still large interference with the interference effect underestimated, but the comparison relationship of different schemes in the test is not affected.

TABLE 8
Verification results of interference effects

| Serial | Interference effects | | | | |
|---|---|---|---|---|---|
| number | HDL-P | LDL-P | Lpa-P | IDL-P | VLDL-P |
| Control | 6.6% | 32.4% | 18.5% | 68.6% | 88.7% |
| Formula | | | | | |
| Formula 1 | 1.3% | 4.6% | 4.3% | 4.8% | 14.0% |
| Formula 2 | 0.9% | 6.6% | 7.1% | 5.0% | 13.1% |
| Formula 3 | 3.7% | 2.6% | 3.7% | 6.5% | 6.1% |
| Formula 4 | 4.2% | 4.8% | 4.2% | 10.6% | 6.7% |
| Formula 5 | 5.3% | 2.5% | 4.0% | 6.2% | 13.9% |
| Formula 6 | 3.5% | 7.0% | 7.3% | 3.2% | 9.5% |
| Formula 7 | 0.9% | 4.4% | 5.5% | 11.2% | 7.0% |
| Formula 8 | 2.9% | 4.2% | 7.5% | 8.3% | 8.2% |
| Formula 9 | 2.0% | 2.5% | 4.6% | 3.7% | 9.2% |
| Formula 10 | 0.2% | 1.9% | 7.4% | 7.3% | 6.5% |
| Formula 11 | 2.7% | 5.3% | 7.1% | 2.9% | 6.2% |
| Formula 12 | 4.0% | 3.7% | 4.6% | 3.6% | 9.6% |
| Formula 13 | 5.8% | 4.9% | 4.0% | 3.0% | 7.2% |
| Formula 14 | −0.6% | 2.1% | 3.6% | 3.1% | 10.0% |
| Formula 15 | 3.2% | 1.1% | 0.0% | 8.1% | 6.7% |
| Formula 16 | 3.7% | 4.4% | 8.1% | 8.9% | 7.1% |
| Formula 17 | 0.2% | 0.3% | 3.0% | 8.1% | 11.3% |
| Formula 18 | −0.1% | 0.9% | 4.8% | 6.4% | 12.1% |
| Formula 19 | 3.5% | 0.1% | 4.3% | 5.8% | 7.4% |
| Formula 20 | 4.0% | 1.9% | 8.1% | 7.4% | 13.4% |
| Formula 21 | 4.6% | 7.8% | 2.0% | 5.5% | 12.8% |
| Formula 22 | −2.0% | 2.9% | 4.5% | 3.3% | 7.5% |
| Formula 23 | 1.2% | 1.8% | 2.5% | 5.0% | 10.8% |
| Formula 24 | −0.2% | 1.7% | 2.8% | 10.4% | 11.6% |
| Formula 25 | 5.2% | 5.1% | 4.1% | 8.2% | 6.1% |
| Formula 26 | 3.0% | 5.4% | 6.6% | 7.7% | 11.3% |
| Formula 27 | 5.9% | 3.3% | 6.6% | 9.9% | 9.1% |
| Formula 28 | 5.9% | 1.4% | 1.2% | 11.1% | 8.8% |
| Formula 29 | −0.8% | 6.9% | 7.6% | 9.3% | 9.2% |
| Formula 30 | 3.0% | 0.0% | 7.7% | 8.8% | 7.8% |
| Formula 31 | 1.0% | 1.5% | 0.3% | 10.2% | 6.7% |
| Formula 32 | 3.9% | 4.6% | 2.4% | 9.1% | 7.7% |
| Formula 33 | 2.7% | 2.4% | 7.1% | 5.1% | 7.4% |
| Formula 34 | −1.3% | 7.5% | 3.6% | 9.7% | 12.6% |
| Formula 35 | 1.2% | 4.5% | 6.8% | 6.0% | 6.2% |
| Formula 36 | 5.5% | 0.2% | 6.2% | 6.7% | 6.7% |
| Formula 37 | 0.5% | 7.3% | 4.2% | 10.4% | 10.5% |
| Formula 38 | −1.7% | 2.0% | 8.0% | 7.2% | 14.1% |
| Formula 39 | 2.4% | 7.5% | 3.0% | 8.6% | 10.5% |
| Formula 40 | 2.9% | 7.8% | 2.7% | 8.6% | 10.2% |
| Formula 41 | 0.7% | 0.7% | 2.4% | 6.7% | 13.3% |
| Formula 42 | 1.4% | 1.8% | 3.5% | 4.6% | 7.5% |
| Formula 43 | −0.4% | 4.0% | 0.4% | 5.8% | 11.3% |
| Formula 44 | −1.1% | 3.9% | 7.6% | 6.8% | 7.5% |
| Formula 45 | 3.4% | 6.3% | 2.4% | 9.6% | 7.3% |
| Formula 46 | 3.4% | 3.2% | 7.9% | 11.2% | 12.9% |
| Formula 47 | 3.2% | 3.3% | 7.4% | 7.2% | 9.9% |
| Formula 48 | 2.3% | 0.8% | 0.3% | 6.5% | 9.0% |
| Formula 49 | −1.2% | 3.2% | 7.1% | 9.8% | 7.7% |
| Formula 50 | 5.2% | 2.6% | 8.1% | 11.2% | 11.8% |
| Formula 51 | −1.8% | 6.0% | 6.6% | 9.3% | 8.2% |
| Formula 52 | −1.4% | 7.6% | 0.8% | 9.4% | 8.6% |
| Formula 53 | −1.8% | 5.8% | 5.9% | 4.1% | 10.7% |
| Formula 54 | 2.6% | 1.0% | 3.1% | 7.3% | 8.3% |
| Formula 55 | 3.8% | 3.2% | 0.0% | 3.5% | 14.0% |
| Formula 56 | −0.1% | 2.3% | 4.4% | 2.9% | 8.1% |
| Formula 57 | 1.8% | 3.6% | 3.4% | 7.8% | 6.5% |
| Formula 58 | 5.1% | 0.8% | 6.2% | 5.3% | 12.4% |
| Formula 59 | 1.8% | 5.9% | 5.8% | 3.1% | 11.5% |
| Formula 60 | 2.4% | 3.8% | 4.2% | 3.6% | 6.5% |
| Formula 61 | 5.0% | 4.6% | 4.3% | 8.8% | 9.9% |
| Formula 62 | 5.7% | 7.9% | 7.1% | 7.4% | 12.4% |
| Formula 63 | 4.6% | 6.0% | 4.7% | 5.0% | 14.0% |
| Formula 54 | −0.4% | 0.9% | 1.0% | 7.0% | 13.1% |
| Formula 65 | 0.3% | 4.7% | 2.6% | 6.1% | 8.9% |
| Formula 66 | 2.9% | 0.8% | 3.9% | 6.0% | 11.8% |
| Formula 67 | −0.5% | 6.2% | 5.7% | 7.7% | 13.5% |

It can be seen that when no alcohol substance is added to the reagent, LDL-P, Lpa-P, IDL-P and VLDL-P are all highly susceptible to the interference of the high TG sample. When an alcohol substance is added to the reagent, these items are much less susceptible to the interference of the high TG. Hence, the present invention helps to reduce the TG interference at the time of blood lipid particle detection and can provide more precise detection result to the clinics.

### Embodiment 5

Sample Comparison

40 normal TG samples were selected and then detected using different formula reagents to analyze its relevance with the control formula, with the result shown in Table 9.

TABLE 9
Relevance of detection results of different Formulas

| Item | Relevance with control Formula | | | | |
|---|---|---|---|---|---|
| name | HDL-P | LDL-P | Lpa-P | IDL-P | VLDL-P |
| Formula 3 | y = 1.02x + 1.2, | y = 1.01x + 12.2, | y = 0.99x + 4.6, | y = 1.02x + 1.8, | y = 1.01x + 8.5, |
| | r = 0.9971 | r = 0.9976 | r = 0.9872 | r = 0.9916 | r = 0.9869 |
| Formula 10 | y = 1.01x + 2.1, | y = 1.00x + 10.2, | y = 0.98x − 4.1, | y = 1.01x − 2.8, | y = 1.02x + 7.5, |
| | r = 0.9879 | r = 0.9965 | r = 0.9902 | r = 0.9896 | r = 0.9969 |
| Formula 15 | y = 1.03x + 1.6, | y = 1.01x + 9.2, | y = 1.01x − 1.1, | y = 1.01x − 2.1, | y = 1.02x + 3.3, |
| | r = 0.9899 | r = 0.9959 | r = 0.9898 | r = 0.9905 | r = 0.9924 |
| Formula 20 | y = 1.01x + 2.5, | y = 1.01x + 6.3, | y = 1.02x − 8.3, | y = 1.02x − 4.1, | y = 1.02x + 6.2, |
| | r = 0.9878 | r = 0.9982 | r = 0.9886 | r = 0.9915 | r = 0.9974 |
| Formula 26 | y = 1.01x + 3.5, | y = 1.02x + 8.8, | y = 1.01x + 3.8, | y = 1.02x − 3.5, | y = 1.02x + 4.2, |
| | r = 0.9964 | r = 0.9926 | r = 0.9889 | r = 0.9926 | r = 0.9964 |
| Formula 32 | y = 1.00x + 3.1, | y = 1.01x + 8.1, | y = 1.00x + 3.2, | y = 1.02x − 3.2, | y = 1.01x + 7.9, |
| | r = 0.9968 | r = 0.9899 | r = 0.9921 | r = 0.9967 | r = 0.9958 |
| Formula 37 | y = 1.01x + 3.5, | y = 1.02x + 16.3, | y = 1.01x-8.9, | y = 1.02x − 4.9, | y = 1.03x + 6.6, |
| | r = 0.9899 | r = 0.9916 | r = 0.9912 | r = 0.9926 | r = 0.9964 |
| Formula 44 | y = 0.99x + 1.8, | y = 1.01x + 9.2, | y = 1.01x − 1.1, | y = 1.00x − 2.7, | y = 1.01x + 5.3. |
| | r = 0.9891 | r = 0.9951 | r = 0.9968 | r = 0.9899 | r = 0.9921 |
| Formula 49 | y = 1.03x + 2.6, | y = 1.02x + 19.2, | y = 1.04x − 6.9, | y = 0.98x − 2.8, | y = 1.01x + 3.8, |
| | r = 0.9896 | r = 0.9963 | r = 0.9866 | r = 0.9911 | r = 0.9985 |
| Formula 50 | y = 0.99x + 2.6, | y = 0.99x + 11.2, | y = 0.98x − 6.1, | y = 1.01x − 3.8, | y = 0.99x + 7.8, |
| | r = 0.9889 | r = 0.9925 | r = 0.9911 | r = 0.9866 | r = 0.9923 |

It can be seen that for the normal TG sample, compared with control formula, the results of these different formulas have good relevance, namely, addition of an alcohol substance to the reagent does not affect the detection result.

In conclusion, by using the reagent of the present invention, density gradient stratification can be performed on the serum sample effectively so as to perform subsequent detections of the substances such as lipoproteins. The reagent can detect the lipoprotein particle concentration separately, and on the other hand, can also detect the cholesterol sub-components of the lipoproteins at the same time in cooperation with other instruments and reagents, thus combining two processes into one process and saving time and costs. It has high commercial value and can be widely applied to clinical detections and scientific researches.

The above descriptions are made to the preferred embodiments of the present invention and shall not be understood as limiting of the claims. The present invention is not limited to the above embodiments and its specific structure is allowed to have change. Various changes made within the scope of protection of the independent claims of the present invention shall all fall within the scope of protection of the present invention.

1. A reagent for detecting a lipoprotein particle concentration, wherein the reagent comprises the following components: a diluent, a density liquid and a buffer solution, the density liquid contains an alcohol substance with a concentration of 0.1% to 50%, and a volumetric ratio of the diluent to the density liquid to the buffer solution is 1: (35-45): (110-130).

2. The reagent for detecting a lipoprotein particle concentration of claim 1, wherein the number of the carbon atoms contained in the alcohol substance is 1, 2, 3 or 4.

3. The reagent for detecting a lipoprotein particle concentration of claim 1, wherein the alcohol substance is one or more of methanol, ethanol, ethylene glycol, n-propanol, isopropanol, propylene glycol, n-butanol, isobutanol and tertiary butanol.

4. The reagent for detecting a lipoprotein particle concentration of claim 1, wherein the concentration of the alcohol substance is 1% to 5%.

5. The reagent for detecting a lipoprotein particle concentration of claim 1, wherein the density liquid further comprises NaCl and EDTA.

6. The reagent for detecting a lipoprotein particle concentration of claim 1, wherein the diluent is one of a KBr solution, a NaBr solution and a sucrose solution, and a concentration of the diluent is 1.21 g/cm³.

7. The reagent for detecting a lipoprotein particle concentration of claim 1, wherein the buffer solution is one of a tris hydroxymethyl aminomethane hydrochloride buffer solution, a phosphate buffer solution, a 4-(2-hydroxyethyl)piperazine-1-ethanesulfonic acid hemisodium salt buffer solution, a 3-(N-morpholino) ethanesulfonic acid buffer solution, and an N-tris(hydroxymethyl)methyl-2-aminoethanesulfonic acid buffer solution.

8. The reagent for detecting a lipoprotein particle concentration of claim 7, wherein the buffer solution has a pH of 6.0 to 8.0 and a concentration of 20 to 50 mM.

9. A using method of the reagent for detecting a lipoprotein particle concentration of claim 1, wherein it comprises the following steps:
I. diluting a serum sample by using the diluent and fully mixing the serum sample to uniformity to obtain a diluted sample, and adding the density liquid to a centrifugal tube; taking a part of the diluted sample and slowly adding the diluted sample to the centrifugal tube through the bottom of the centrifugal tube to obtain a mixed solution;
II. placing the treated mixed solution centrifugal tube into an ultracentrifuge for centrifugation and obtaining a stratification reagent after the centrifugation, wherein a rotor used in the ultracentrifuge is a vertical rotor;
III. taking out the above stratification reagent and placing the reagent into a blood lipid particle tester for detection, and adding the buffer solution to the blood lipid particle tester; during detection, performing operation as instructed in the use manual of the tester; ranking the centrifuged lipoproteins based on density and enabling the centrifuged lipoproteins to sequentially go through a light scattering detector which records received signals, and finally by using the tester, calculating the particle concentration of the lipoproteins in the sample and thus completing test.

10. The using method of the reagent for detecting a lipoprotein particle concentration of claim 9, wherein in step II, the specific process of the centrifugation is rotation speed 60000 to 70000 rpm, temperature 20 to 25° C., acceleration=6, deceleration=6, and centrifugation time 20 to 30 minutes.
